(19) United States
(12) Patent Application Publication
(10) Pub. No.: US 2022/0169725 A1
(43) Pub. Date: Jun. 2, 2022

(54) BIO-RESPONSIVE ANTIBODY COMPLEXES FOR ENHANCED IMMUNOTHERAPY

(71) Applicant: NORTH CAROLINA STATE UNIVERSITY, Raleigh, NC (US)

(72) Inventors: Zhen GU, Los Angeles, CA (US); Qian CHEN, Raleigh, NC (US)

(21) Appl. No.: 17/437,179

(22) PCT Filed: Mar. 9, 2020
(86) PCT No.: PCT/US2020/021709

§ 371 Date: Sep. 8, 2021

Related U.S. Application Data
(60) Provisional application No. 62/816,017, filed on Mar. 8, 2019.

(51) Int. Cl. C07K 16/28; A61K 45/06; A61K 47/64; A61P 35/00

(52) U.S. Cl. CPC C07K 16/2803; A61K 45/06; A61K 2039/507; A61P 35/00; A61K 47/643

(57) ABSTRACT

Disclosed are bioresponsive protein complexes comprising immune checkpoint blockade inhibitors and methods of their use.

This application claims the benefit of U.S. Provisional Application No. 62/816,017, filed on Mar. 8, 2019, which is incorporated herein by reference in its entirety.

### I. BACKGROUND

Immune checkpoint blockade (ICB) therapy, especially blocking cytotoxic T lymphocyte antigen 4 (CTLA-4), and programmed cell death protein 1/programmed cell death-ligand 1 (PD-1/PD-L1), has achieved exciting clinical progress in many malignancies, including non-small cell lung, melanoma, urothelial carcinoma, renal cell carcinoma, bladder, head and neck cancers. Despite these achievements of ICB in clinic, many challenges remain to be overcome, such as the low objective response rate and systemic side effects. Activated T cells can only induce durable immune responses after CTLA-4 or PD-1/PD-L1 blockade in patients suffering from immunogenic tumors featured with high expression of tumor-associated antigens. In addition, side effects such as autoimmune diseases often occur during the ICB treatment, owing to the off-target binding of antibodies to normal cells. Efforts to promote ICB responses yet avoid severe side effects have become one of the central themes in the field of cancer immunotherapy.

Cancer cells can usually evade the immune system recognition via up-regulation of the integrin-associated protein, also called “don't eat me” signal (CD47). Blocking CD47 will active phagocytic cells to phagocytize cancer cells and promote antigen presentation. These results pave the rationale to presume that CD47 blockade can be used to promote objective response of CTLA-4 or PD-1/PD-L1 blockade. Moreover, engineering bioresponsive immunotherapeutic formations which can respond to the tumor microenvironment (TME) for controlled release of therapeutics is increasingly desired for regulated release of immunomodulatory antibodies, thereby enhancing their retention and efficacy in the tumor and minimizing systemic toxicities. What are needed are new methods and compositions for inhibiting endogenous immunosuppressive signaling.

### II. SUMMARY

Disclosed are methods and compositions related to bioresponsive hydrogel matrixes.

In one aspect, disclosed herein are bioresponsive hydrogel matrixes comprising a CD47/SIRPα inhibitor (such as, for example, Hu5F9-G4, CV1, B6H12, 2D3, CC-90002, and TTI-621) and an immune checkpoint blockade inhibitor (such as, for example, PD-1/PD-L1 inhibitor and/or a CTLA-4/B7-1/2 inhibitor).

Also disclosed herein are bioresponsive hydrogel matrixes of any preceding aspect, wherein the immune checkpoint blockade inhibitor is a PD-1/PD-L1 blockade inhibitor (such as, for example, nivolumab, pembrolizumab, pidilizumab, atezolizumab, avelumab, durvalumab, and BMS-936559).

Also disclosed herein are bioresponsive hydrogel matrixes of any preceding aspect, wherein the immune checkpoint blockade inhibitor is a CTLA-4/B7-1/2 blockade inhibitor (such as, for example, Ipilimumab).

In one aspect, disclosed herein are bioresponsive hydrogel matrixes of any preceding aspect, wherein the hydrogel matrix comprises a reactive oxygen species (ROS) degradable hydrogel (such as, for example, a hydrogel comprising albumin and a bis-N-hydroxy succinimide (NHS) modified 2,2′-[Propane-2,2-diylbis(thio)]diacetic acid (NHS-IE-NHS) cross-linker).

Also disclosed herein are bioresponsive hydrogel matrixes of any preceding aspect, wherein bioresponsive hydrogel matrix comprises an inner core and an outer shell; and wherein the CD47/SIRPa inhibitor is cross-linked to the outer shell and the immune checkpoint inhibitor in cross-linked to the inner core or wherein the CD47/SIRPa inhibitor is cross-linked to the inner core and the immune checkpoint inhibitor in cross-linked to the outer shell.

In one aspect, disclosed herein are methods of treating, preventing, inhibiting, ameliorating, and/or reducing a cancer and/or metastasis in a subject comprising administering to the subject the bioresponsive hydrogel of any preceding aspect. For example, disclosed herein are methods of treating, preventing, inhibiting, and/or reducing a cancer and/or metastasis in a subject comprising administering to the subject a bioresponsive hydrogel comprising a CD47/SIRPα inhibitor (such as, for example, Hu5F9-G4, CV1, B6H12, 2D3, CC-90002, and TTI-621) and an immune checkpoint blockade inhibitor (such as, for example, a PD-1/PD-L1 inhibitor and/or a CTLA-4/B7-1/2 inhibitor).

### III. BRIEF DESCRIPTION OF THE DRAWINGS

The accompanying drawings, which are incorporated in and constitute a part of this specification, illustrate several embodiments and together with the description illustrate the disclosed compositions and methods.

FIGS. 1A, 1B, 1C, 1D, 1E, 1F, and 1G show schematic and characterization of ROS-responsive aPD1@aCD47 protein complex. FIG. 1A shows a schematic illustration showing the synergistic immunotherapy using the ROS-sensitive complexes for controlled sequential release of aCD47 and aPD1 in the tumor microenvironment. FIGS. 1B and 1C show the average hydrodynamic size of aPD1 core (1B) and aPD1@aCD47 complex (1C) determined by DLS. Inset: TEM images of aPD1 core (B) and aPD1@aCD47 complex (1C) (scale bar: 200 nm). FIG. 1D shows scanning TEM (STEM) images of aPD1@aCD47 complex showing the gadolinium labeled aCD47 (green) and calcium labeled aPD1 (Red) (scale bar: 100 nm). FIG. 1E shows the degradation behaviors of aPD1@aCD47 complexes in PBS with and without H₂O₂ (0.5 mM) measured by DLS. Inset: TEM image of aPD1@aCD47 complexes in PBS with H₂O₂ (scale bar: 100 nm). FIG. 1F shows the release profiles of aCD47 and aPD1 from complex dispersed in PBS with or without H₂O₂ (0.5 mM). Data are presented as mean±s.e.m. (n=3). FIG. 1G shows the H₂O₂ scavenging test using the complexes in PBS containing H₂O₂. Data are presented as mean±s.e.m. (n=3).

FIGS. 2A, 2B, 2C, 2D, 2E, 2F, 2G, and 2H show ROS-responsive protein complexes for scavenging ROS in the TME to reverse the immunosuppressive environment. FIGS. 2A and 2B show ROS levels in the tumor collected from mice with or without complex treatment were measured using the CELLROX® deep red reagent by flow cytometric analyses (2A) and confocal fluorescence imaging (2B) on day 5. a.u., arbitrary unit. FIG. 2C shows the expression of NF-κB p65 and MMP2 in B16F10 tumors analyzed by Western blotting. FIG. 2D shows the percentage of CD45⁺ cells in B16F10 tumors analyzed by flow cytometry. FIG. 2E shows the percentage of M2-like macrophages (CD206^{hi}F4/80⁺CD11b⁺) in B16F10 tumors analyzed by flow cytometry. FIG. 2F shows the percentage of CD4⁺Foxp3⁺ T cells in B16F10 tumors analyzed by flow cytometry. FIG. 2G shows the percentage of CD8⁺ T cells in B16F10 tumors analyzed by flow cytometry. Data are presented as mean±s.e.m. (n=4). FIG. 2H shows the schematic illustration showing the various immune responses after ROS-sensitive complex treatment. Statistical significance was calculated via two-tailed Student's t-test. P value: *P<0.05;**P<0.01;***P<0.005.

FIGS. 3A and 3B show the absolute percentage of CD3⁺ (3A) and CD4⁺ T cells (3B) within tumors after the ROS-responsive complex treatment. Data are presented as mean±s.e.m. (n=4). Statistical significance was calculated via two-tailed Student's t-test. P value: *P<0.05; **P<0.01;***P<0.005.

FIGS. 4A, 4B, 4C, 4D, and 4E show CD47 blockade for increasing phagocytosis of cancer cells and activating phagocytic immune cells. FIG. 4A shows representative confocal images showing that aCD47 treatment resulted in robust phagocytosis of red fluorescently labeled B16F10 cells by green fluorescently labeled BMDMs (Scale bar, 50 μm). FIG. 4B shows the phagocytosis of cancer cells by BMDMs determined by flow cytometry. Data are presented as mean±s.e.m. (n=3). FIG. 4C shows percentage of CD11c⁺ DCs gating on CD45⁺ cells in the tumor after CD47 blockade. FIGS. 4D and 4E show the percentage of CD80⁺CD86⁺ DCs (4D) and CD103⁺ DCs (4E) gating on CD45⁺CD11c⁺ cells in the tumor after CD47 blockade. Data are presented as mean±s.e.m. (n=4). Statistical significance was calculated via two-tailed Student's t-test. P value: *P<0.05; **P<0.01; ***P<0.005.

FIGS. 5A, 5B, and 5C show the retention behavior of intratumorally injected protein complex. FIGS. 5A and 5B show In vivo fluorescence imaging to show the retention of aCD47 (5A) and aPD1 (5B) in the tumor at different time points after injection of free antibodies or aPD1@aCD47 complexes. FIG. 5C shows confocal immunofluorescence images of tumors collected from mice treated with free antibodies or aPD1@aCD47 complexes at different time points (Scale bar, 200 μm). Red and green signals indicate aPD1 and aCD47, respectively.

FIGS. 6A, 6B, 6C, 6D, 6E, 6F, and 6G show protein complex-mediated checkpoint blockade for inhibiting B16F10 tumor growth in vivo. FIG. 6A shows In vivo bioluminescence imaging of B16F10 tumor after different treatments. Four representative mice are shown per group. FIGS. 6B and 6C show individual (6B) and average (6C) tumor growth curves in different groups. Data are presented as mean±s.e.m. (n=6). FIGS. 6D, 6E, and 6F show the absolute percentage of CD3⁺ T cells (6D), CD4⁺ T cells (6E) and CD8⁺ T cells (6F) in the tumor after different treatments. Data are presented as mean±s.e.m. (n=4). FIG. 6G show representative flow cytometric analyses of T cell infiltration in the tumor. Statistical significance was calculated via one-way ANOVA with a Tukey post-hoc test. P value: *P<0.05; **P<0.01; ***P<0.001.

FIGS. 7A, 7B, 7C, 7D, 7E, 7F, 7G, and 7H show protein complex-mediated checkpoint blockade for inhibiting distant tumor growth. FIG. 7A shows a schematic illustrating aPD1@aCD47 complex treatment in inhibiting cancer metastasis. Tumor on the right side was designated as “primary tumor” with aPD1@aCD47 complex treatment, and tumor on the left side was designated as “metastatic tumor” without any treatment. FIG. 7B shows In vivo bioluminescence imaging of B16F10 tumor after local injection with aPD1@aCD47 complex. Four representative mice are shown per group, respectively. FIGS. 7C and 7D show left and right tumor growth curves (7C) and weights (7D) in untreated and treated mice. Data are presented as mean±s.e.m. (n=6). (e-h) Representative flow cytometric analysis of T cell infiltration in the tumor (7E) and absolute percentage of the CD3⁺ T cells (7F), CD4⁺ T cells (7G) and CD8⁺ T cells (7H) in the tumor after different treatments. Data are presented as mean±s.e.m. (n=4). Statistical significance was calculated via one-way ANOVA with a Tukey post-hoc test. P value: *P<0.05; **P<0.01; ***P<0.001.

### IV. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

The term “subject” is defined herein to include animals such as mammals, including, but not limited to, primates (e.g., humans), cows, sheep, goats, horses, dogs, cats, rabbits, rats, mice and the like. In some embodiments, the subject is a human.

“Administration” to a subject includes any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, parenteral (e.g., subcutaneous, intravenous, intramuscular, intra-articular, intra-synovial, intrasternal, intrathecal, intraperitoneal, intrahepatic, intralesional, and intracranial injections or infusion techniques), and the like. “Concurrent administration”, “administration in combination”, “simultaneous administration” or “administered simultaneously” as used herein, means that the compounds are administered at the same point in time or essentially immediately following one another. In the latter case, the two compounds are administered at times sufficiently close that the results observed are indistinguishable from those achieved when the compounds are administered at the same point in time. “Systemic administration” refers to the introducing or delivering to a subject an agent via a route which introduces or delivers the agent to extensive areas of the subject's body (e.g. greater than 50% of the body), for example through entrance into the circulatory or lymph systems. By contrast, “local administration” refers to the introducing or delivery to a subject an agent via a route which introduces or delivers the agent to the area or area immediately adjacent to the point of administration and does not introduce the agent systemically in a therapeutically significant amount. For example, locally administered agents are easily detectable in the local vicinity of the point of administration, but are undetectable or detectable at negligible amounts in distal parts of the subject's body. Administration includes self-administration and the administration by another.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

A “control” is an alternative subject or sample used in an experiment for comparison purposes. A control can be “positive” or “negative.”

“Controlled release” or “sustained release” refers to release of an agent from a given dosage form in a controlled fashion in order to achieve the desired pharmacokinetic profile in vivo. An aspect of “controlled release” agent delivery is the ability to manipulate the formulation and/or dosage form in order to establish the desired kinetics of agent release.

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

A “decrease” can refer to any change that results in a smaller gene expression, protein expression, amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

The terms “prevent,” “preventing,” “prevention,” and grammatical variations thereof as used herein, refer to a method of partially or completely delaying or precluding the onset or recurrence of a disease and/or one or more of its attendant symptoms or barring a subject from acquiring or reacquiring a disease or reducing a subject's risk of acquiring or reacquiring a disease or one or more of its attendant symptoms.

“Pharmaceutically acceptable” component can refer to a component that is not biologically or otherwise undesirable, i.e., the component may be incorporated into a pharmaceutical formulation of the invention and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Polymer” refers to a relatively high molecular weight organic compound, natural or synthetic, whose structure can be represented by a repeated small unit, the monomer. Non-limiting examples of polymers include polyethylene, rubber, cellulose. Synthetic polymers are typically formed by addition or condensation polymerization of monomers. The term “copolymer” refers to a polymer formed from two or more different repeating units (monomer residues). By way of example and without limitation, a copolymer can be an alternating copolymer, a random copolymer, a block copolymer, or a graft copolymer. It is also contemplated that, in certain aspects, various block segments of a block copolymer can themselves comprise copolymers. The term “polymer” encompasses all forms of polymers including, but not limited to, natural polymers, synthetic polymers, homopolymers, heteropolymers or copolymers, addition polymers, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. Compositions

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular bioresponsive hydrogel matrix is disclosed and discussed and a number of modifications that can be made to a number of molecules including the bioresponsive hydrogel matrix is discussed, specifically contemplated is each and every combination and permutation of bioresponsive hydrogel matrix and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

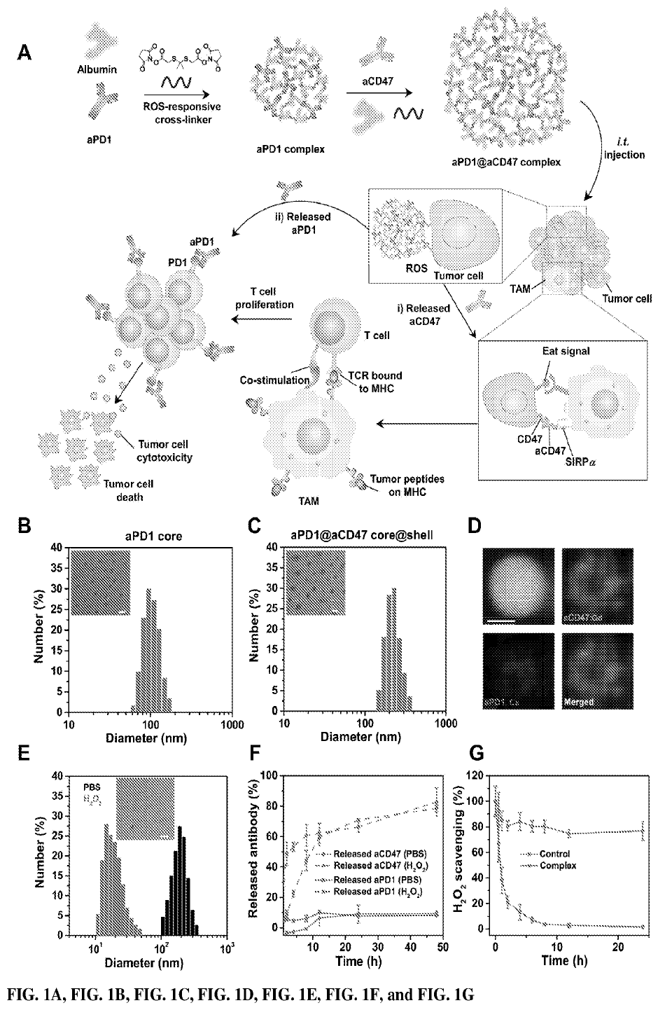

Herein, an albumin-based complex with anti-PD-1 (aPD1) in the core and anti-CD47 (aCD47) in the shell (aPD1@aCD47 complex) was engineered using reactive oxygen species (ROS) responsive linkers, for combination therapy. It was shown herein that, in the ROS-enriched TME, ROS-responsive aPD1@aCD47 complexes can first sustainably release aCD47 from the outer shell, to activate the recognition of cancer cells by the innate immune system and boost T cell responses. Then, the subsequently released aPD1 can exert the PD1 blockade to effectively increase alloreactive T cells to attack the cancer cells. Moreover, the ROS-responsive complex not only serves as a reservoir for the controlled release of antibodies, but also modulates ROS levels in the TME (FIG. 1A). ROS, an important signaling messenger in the immune system, is closely associated with the immunosuppressive responses, promoting tumor development and progression. Thus, the ROS-degradable complexes can promote effective antitumor immune responses by a controlled sequential release of aCD47 and aPD1, together with downregulation of ROS sensitive signals within the TME.

Disclosed herein disclosed herein are bioresponsive hydrogel matrixes comprising a CD47/SIRPα inhibitor (such as, for example, Hu5F9-G4, CV1, B6H12, 2D3, CC-90002, and TTI-621) and an immune checkpoint blockade inhibitor (such as, for example, PD-1/PD-L1 inhibitor and/or a CTLA-4/B7-1/2 inhibitor).

In one aspect, the blockade inhibitor that can be used in the disclosed bioresponsive hydrogel matrixes can be any inhibitor of an immune checkpoint blockade inhibitor, such as for example, a PD-1/PD-L1 blockade inhibitor, and/or a CTLA-4/B7-1/2 blockade inhibitor (such as for example, Ipilimumab). Examples, of PD-1/PD-L1 blockade inhibitors for use in the disclosed bioresponsive hydrogel matrixes can include any PD-1/PD-L1 blockade inhibitor known in the art, including, but not limited to nivolumab, pembrolizumab, pidilizumab, atezolizumab, avelumab, durvalumab, and BMS-936559).

As noted herein, the disclosed bioresponsive hydrogel matrixes utilize a CD47/Signal Regulator Protein alpha (SIRPα) inhibitor (such as for example, Hu5F9-G4, CV1, B6H12, 2D3, CC-90002, and/or TTI-621) inhibitor to sensitize the subject to immune checkpoint inhibition therapy. It is understood and herein contemplated that the CD47/SIRPα inhibitor used in the disclosed bioresponsive hydrogel matrixes can comprise any known CD47/SIRPα inhibitor, including, but not limited to Hu5F9-G4, CV1, B6H12, 2D3, CC-90002, and/or TTI-621.

To facilitate these functions, the bioresponsive hydrogel matrix can be engineered as a polymer. “Polymer” refers to a relatively high molecular weight organic compound, natural or synthetic, whose structure can be represented by a repeated small unit, the monomer. Non-limiting examples of polymers include polyethylene, rubber, cellulose. Synthetic polymers are typically formed by addition or condensation polymerization of monomers. The term “copolymer” refers to a polymer formed from two or more different repeating units (monomer residues). By way of example and without limitation, a copolymer can be an alternating copolymer, a random copolymer, a block copolymer, or a graft copolymer. It is also contemplated that, in certain aspects, various block segments of a block copolymer can themselves comprise copolymers. The term “polymer” encompasses all forms of polymers including, but not limited to, natural polymers, synthetic polymers, homopolymers, heteropolymers or copolymers, addition polymers, etc. In one aspect, the gel matrix can comprise copolymers, block copolymers, diblock copolymers, and/or triblock copolymers.

In one aspect, the bioresponsive hydrogel matrix can comprise a biocompatible polymer (such as, for example, methacrylated hyaluronic acid (m-HA)). In one aspect, biocompatible polymer can be crosslinked. Such polymers can also serve to slowly release the adipose browning agent and/or fat modulating agent into tissue. As used herein biocompatible polymers include, but are not limited to polysaccharides; hydrophilic polypeptides; poly(amino acids) such as poly-L-glutamic acid (PGS), gamma-polyglutamic acid, poly-L-aspartic acid, poly-L-serine, or poly-L-lysine; polyalkylene glycols and polyalkylene oxides such as polyethylene glycol (PEG), polypropylene glycol (PPG), and poly(ethylene oxide) (PEO); poly(oxyethylated polyol); poly(olefinic alcohol); polyvinylpyrrolidone); poly(hydroxyalkylmethacrylamide); poly(hydroxyalkylmethacrylate); poly(saccharides); poly(hydroxy acids); poly(vinyl alcohol), polyhydroxyacids such as poly(lactic acid), poly (gly colic acid), and poly (lactic acid-co-glycolic acids); polyhydroxyalkanoates such as poly3-hydroxybutyrate or poly4-hydroxybutyrate; polycaprolactones; poly(orthoesters); polyanhydrides; poly(phosphazenes); poly(lactide-co-caprolactones); polycarbonates such as tyrosine polycarbonates; polyamides (including synthetic and natural polyamides), polypeptides, and poly(amino acids); polyesteramides; polyesters; poly(dioxanones); poly(alkylene alkylates); hydrophobic polyethers; polyurethanes; polyetheresters; polyacetals; polycyanoacrylates; polyacrylates; polymethylmethacrylates; polysiloxanes; poly(oxyethylene)/poly(oxypropylene) copolymers; polyketals; polyphosphates; polyhydroxyvalerates; polyalkylene oxalates; polyalkylene succinates; poly(maleic acids), as well as copolymers thereof. Biocompatible polymers can also include polyamides, polycarbonates, polyalkylenes, polyalkylene glycols, polyalkylene oxides, polyalkylene terepthalates, polyvinyl alcohols (PVA), methacrylate PVA (m-PVA), polyvinyl ethers, polyvinyl esters, polyvinyl halides, polyvinylpyrrolidone, polyglycolides, polysiloxanes, polyurethanes and copolymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly (methyl methacrylate), poly(ethylmethacrylate), poly(butylmethacrylate), poly(isobutylmethacrylate), poly(hexlmethacrylate), poly(isodecylmethacrylate), poly(lauryl methacrylate), poly (phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), poly(vinyl acetate, poly vinyl chloride polystyrene and polyvinylpryrrolidone, derivatives thereof, linear and branched copolymers and block copolymers thereof, and blends thereof. Exemplary biodegradable polymers include polyesters, poly(ortho esters), poly(ethylene amines), poly(caprolactones), poly(hydroxybutyrates), poly(hydroxyvalerates), polyanhydrides, poly(acrylic acids), polyglycolides, poly(urethanes), polycarbonates, polyphosphate esters, polyphospliazenes, derivatives thereof, linear and branched copolymers and block copolymers thereof, and blends thereof.

In some embodiments the bioresponsive hydrogel matrix contains biocompatible and/or biodegradable polyesters or polyanhydrides such as poly(lactic acid), poly(glycolic acid), and poly(lactic-co-glycolic acid). The bioresponsive hydrogel matrixes can contain one more of the following polyesters: homopolymers including glycolic acid units, referred to herein as “PGA”, and lactic acid units, such as poly-L-lactic acid, poly-D-lactic acid, poly-D,L-lactic acid, poly-L-lactide, poly-D-lactide, and poly-D,L-lactide5 collectively referred to herein as “PLA”, and caprolactone units, such as poly(e-caprolactone), collectively referred to herein as “PCL”; and copolymers including lactic acid and glycolic acid units, such as various forms of poly(lactic acid-co-glycolic acid) and poly(lactide-co-glycolide) characterized by the ratio of lactic acid:glycolic acid, collectively referred to herein as “PLGA”; and polyacrylates, and derivatives thereof. Exemplary polymers also include copolymers of polyethylene glycol (PEG) and the aforementioned polyesters, such as various forms of PLGA-PEG or PLA-PEG copolymers, collectively referred to herein as “PEGylated polymers”. In certain embodiments, the PEG region can be covalently associated with polymer to yield “PEGylated polymers” by a cleavable linker. In one aspect, the polymer comprises at least 60, 65, 70, 75, 80, 85, 89, 90, 91, 92, 93, 94, 95, 96, 97, 98, or 99 percent acetal pendant groups.

The triblock copolymers disclosed herein comprise a core polymer such as, example, polyethylene glycol (PEG), polyvinyl acetate, polyvinyl alcohol, polyvinyl pyrrolidone (PVP), polyethyleneoxide (PEO), poly(vinyl pyrrolidone-co-vinyl acetate), polymethacrylates, polyoxyethylene alkyl ethers, polyoxyethylene castor oils, polycaprolactam, polylactic acid, polyglycolic acid, poly(lactic-glycolic) acid, poly(lactic co-glycolic) acid (PLGA), cellulose derivatives, such as hydroxymethylcellulose, hydroxypropylcellulose and the like. In one aspect, the core polymer can be flanked by polypeptide blocks.

Examples of diblock copolymers that can be used in the micelles disclosed herein comprise a polymer such as, example, polyethylene glycol (PEG), polyvinyl acetate, polyvinyl alcohol (PVA), polyvinyl pyrrolidone (PVP), polyethyleneoxide (PEO), poly(vinyl pyrrolidone-co-vinyl acetate), polymethacrylates, polyoxyethylene alkyl ethers, polyoxyethylene castor oils, polycaprolactam, polylactic acid, polyglycolic acid, poly(lactic-glycolic) acid, poly(lactic co-glycolic) acid (PLGA)

It is understood and herein contemplated that the bioresponsive hydrogel matrix can be designed to be bioresponsive to the microenvironment of the tumor and release the CD47/SIRPα inhibitor (such as, for example, Hu5F9-G4, CV1, B6H12, 2D3, CC-90002, and TTI-621) and the immune checkpoint blockade inhibitor (such as, for example, PD-1/PD-L1 inhibitor and/or a CTLA-4/B7-1/2 inhibitor), and any further anti-cancer agents into the tumor microenvironment upon exposure to factors within the microenvironment such as, for example reactive oxygen species, including, but not limited to peroxides (for example hydrogen peroxide), superoxide, hydroxyl radical, and singlet oxygen; the presence of acidity; redox potential (glutathione (GSH)); specific tumor-associated enzymes; hypoxia; and adenosine-5′-triphosphate (ATP). Thus, in one aspect, disclosed herein the bioresponsive hydrogel matrixes disclosed herein comprises a bioresponsive scaffold that releases the CD47/SIRPα inhibitor (such as, for example, Hu5F9-G4, CV1, B6H12, 2D3, CC-90002, and TTI-621) and the immune checkpoint blockade inhibitor (such as, for example, PD-1/PD-L1 inhibitor and/or a CTLA-4/B7-1/2 inhibitor), and/or further anti-cancer agent into a tumor microenvironment upon exposure to factors within the microenvironment (such as, for example, a reactive oxygen species (ROS) degradable hydrogel). In one aspect, the hydrogel can comprise crosslinked polyvinyl alcohol (PVA) and N¹-(4-boronobenzyl)-N³-(4-boronophenyl)-N¹,N¹,N³,N³-tetramethylpropane-1,3-diaminium (TSPBA). In one aspect, the ROS-responsive hydrogel can be obtained by crosslinking poly (vinyl alcohol) (PVA) with a ROS-labile linker: N¹-(4-boronobenzyl)-N³-(4-boronophenyl)-N¹,N¹,N³,N³-tetramethylpropane-1,3-diaminium (TSPBA), which was synthesized via quaternization reaction of N¹,N¹,N³,N³-tetramethylpropane-1,3-diamine with an excess of 4-(bromomethyl) phenylboronic acid. TSPBA contains two phenylboronic acids that complex with multiple diols on PVA. The TSPBA can be oxidized and hydrolyzed when exposed to H₂O₂ in the tumor microenvironment, leading to the dissociation of the polymeric scaffold and the release of PVA and payloads. In another aspect, the hydrogel can comprise albumin cross-linked to both the CD47/SIRPα inhibitor and the immune checkpoint blockade inhibitor via a bis-N-hydroxy succinimide (NHS) modified 2,2′-[Propane-2,2-diylbis(thio)]diacetic acid (NHS-IE-NHS) cross-linker

In one aspect, the CD47/SIRPα inhibitor (such as, for example, Hu5F9-G4, CV1, B6H12, 2D3, CC-90002, and TTI-621) and the immune checkpoint blockade inhibitor (such as, for example, PD-1/PD-L1 inhibitor and/or a CTLA-4/B7-1/2 inhibitor) can be arranged in the bioresponsive hydrogel matrix in a core-shell structure to facilitate the sequential release of the CD47/SIRPα inhibitor and the immune checkpoint blockade inhibitor such that the core bound inhibitor is released into the tumor microenvironment after following the release of the shell bound inhibitor. Thus, in one aspect, disclosed herein are bioresponsive hydrogel matrixes wherein bioresponsive hydrogel matrix comprises an inner core and an outer shell; and wherein the CD47/SIRPa inhibitor is cross-linked to the outer shell and the immune checkpoint inhibitor in cross-linked to the inner core or wherein the CD47/SIRPa inhibitor is cross-linked to the inner core and the immune checkpoint inhibitor in cross-linked to the outer shell.

Anti-cancer agents that can be used in the disclosed bioresponsive hydrogel matrixes can comprise any anti-cancer agent known in the art, the including, but not limited to Abemaciclib, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin Chlorambucil), Amifostine, Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avastin (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Campath (Alemtuzumab), Camptosar, (Irinotecan Hydrochloride), Capecitabine, CAPDX, Carac (Fluorouracil—Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil—Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista, (Raloxifene Hydrochloride), Evomela (Melphalan Hydrochloride), Exemestane, 5-FU (Fluorouracil Injection), 5-FU (Fluorouracil—Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil—Topical), Fluorouracil Injection, Fluorouracil—Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and, Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq, (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil—Topical), Topotecan Hydrochloride, Toremifene, Torisel (Temsirolimus), Tositumomab and Iodine I 131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and/or Zytiga (Abiraterone Acetate).

1. Antibodies

### (1) Antibodies Generally

The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof are also disclosed. The antibodies can be tested for their desired activity using the in vitro assays described herein, or by analogous methods, after which their in vivo therapeutic and/or prophylactic activities are tested according to known clinical testing methods. There are five major classes of human immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. One skilled in the art would recognize the comparable classes for mouse. The heavy chain constant domains that correspond to the different classes of immunoglobulins are called alpha, delta, epsilon, gamma, and mu, respectively.

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity.

The disclosed monoclonal antibodies can be made using any procedure which produces mono clonal antibodies. For example, disclosed monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro.

The monoclonal antibodies may also be made by recombinant DNA methods. DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

As used herein, the term “antibody or fragments thereof” encompasses chimeric antibodies and hybrid antibodies, with dual or multiple antigen or epitope specificities, and fragments, such as F(ab′)2, Fab′, Fab, Fv, scFv, and the like, including hybrid fragments. Thus, fragments of the antibodies that retain the ability to bind their specific antigens are provided. Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

Also included within the meaning of “antibody or fragments thereof” are conjugates of antibody fragments and antigen binding proteins (single chain antibodies).

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

### (2) Human Antibodies

The disclosed human antibodies can be prepared using any technique. The disclosed human antibodies can also be obtained from transgenic animals. For example, transgenic, mutant mice that are capable of producing a full repertoire of human antibodies, in response to immunization, have been described (see, e.g., Jakobovits et al., Proc. Natl. Acad. Sci. USA, 90:2551-255 (1993); Jakobovits et al., Nature, 362:255-258 (1993); Bruggermann et al., Year in Immunol., 7:33 (1993)). Specifically, the homozygous deletion of the antibody heavy chain joining region (J(H)) gene in these chimeric and germ-line mutant mice results in complete inhibition of endogenous antibody production, and the successful transfer of the human germ-line antibody gene array into such germ-line mutant mice results in the production of human antibodies upon antigen challenge. Antibodies having the desired activity are selected using Env-CD4-co-receptor complexes as described herein.

### (3) Humanized Antibodies

Antibody humanization techniques generally involve the use of recombinant DNA technology to manipulate the DNA sequence encoding one or more polypeptide chains of an antibody molecule. Accordingly, a humanized form of a non-human antibody (or a fragment thereof) is a chimeric antibody or antibody chain (or a fragment thereof, such as an sFv, Fv, Fab, Fab′, F(ab′)2, or other antigen-binding portion of an antibody) which contains a portion of an antigen binding site from a non-human (donor) antibody integrated into the framework of a human (recipient) antibody.

To generate a humanized antibody, residues from one or more complementarity determining regions (CDRs) of a recipient (human) antibody molecule are replaced by residues from one or more CDRs of a donor (non-human) antibody molecule that is known to have desired antigen binding characteristics (e.g., a certain level of specificity and affinity for the target antigen). In some instances, Fv framework (FR) residues of the human antibody are replaced by corresponding non-human residues. Humanized antibodies may also contain residues which are found neither in the recipient antibody nor in the imported CDR or framework sequences. Generally, a humanized antibody has one or more amino acid residues introduced into it from a source which is non-human. In practice, humanized antibodies are typically human antibodies in which some CDR residues and possibly some FR residues are substituted by residues from analogous sites in rodent antibodies. Humanized antibodies generally contain at least a portion of an antibody constant region (Fc), typically that of a human antibody (Jones et al., Nature, 321:522-525 (1986), Reichmann et al., Nature, 332:323-327 (1988), and Presta, Curr. Opin. Struct. Biol., 2:593-596 (1992)).

Methods for humanizing non-human antibodies are well known in the art. For example, humanized antibodies can be generated according to the methods of Winter and co-workers (Jones et al., Nature, 321:522-525 (1986), Riechmann et al., Nature, 332:323-327 (1988), Verhoeyen et al., Science, 239:1534-1536 (1988)), by substituting rodent CDRs or CDR sequences for the corresponding sequences of a human antibody. Methods that can be used to produce humanized antibodies are also described in U.S. Pat. No. 4,816,567 (Cabilly et al.), U.S. Pat. No. 5,565,332 (Hoogenboom et al.), U.S. Pat. No. 5,721,367 (Kay et al.), U.S. Pat. No. 5,837,243 (Deo et al.), U.S. Pat. No. 5,939,598 (Kucherlapati et al.), U.S. Pat. No. 6,130,364 (Jakobovits et al.), and U.S. Pat. No. 6,180,377 (Morgan et al.).

### 2. Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

a) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

b) Therapeutic Uses

Effective dosages and schedules for administering the compositions may be determined empirically, and making such determinations is within the skill in the art. The dosage ranges for the administration of the compositions are those large enough to produce the desired effect in which the symptoms of the disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

### C. Method of Treating Cancer

In one aspect, disclosed herein are methods of treating, preventing, inhibiting, and/or reducing a cancer and/or metastasis in a subject comprising administering to the subject any of the bioresponsive hydrogel matrixes disclosed herein. For example, disclosed herein are methods of treating, preventing, inhibiting, and/or reducing a cancer and/or metastasis (such as, for example, a cancer with low PD-L1 expression or a non-immunogenic cancer selected from the group consisting of melanoma, non-small cell lung carcinoma, renal cancer, head and neck cancer, and/or bladder cancer) in a subject comprising administering to the subject a bioresponsive hydrogel matrix comprising a CD47/SIRPα inhibitor (such as, for example, Hu5F9-G4, CV1, B6H12, 2D3, CC-90002, and TTI-621) and an immune checkpoint blockade inhibitor (such as, for example, a PD-1/PD-L1 inhibitor and/or a CTLA-4/B7-1/2 inhibitor).

“Treat,” “treating,” “treatment,” and grammatical variations thereof as used herein, include the administration of a composition with the intent or purpose of partially or completely preventing, delaying, curing, healing, alleviating, relieving, altering, remedying, ameliorating, improving, stabilizing, mitigating, and/or reducing the intensity or frequency of one or more a diseases or conditions, a symptom of a disease or condition, or an underlying cause of a disease or condition. Treatments according to the invention may be applied preventively, prophylactically, pallatively or remedially. Prophylactic treatments are administered to a subject prior to onset (e.g., before obvious signs of cancer), during early onset (e.g., upon initial signs and symptoms of cancer), or after an established development of cancer. Prophylactic administration can occur for day(s) to years prior to the manifestation of symptoms of an infection.

In one aspect, either the immune checkpoint blockade inhibitor used in the disclosed methods of treating, preventing, inhibiting, and/or reducing a cancer and/or metastasis in a subject comprises any inhibitor of an immune checkpoint known in the art, such as for example, a PD-1/PD-L1 blockade inhibitor, or a CTLA-4/B7-1/2 blockade inhibitor (such as for example, Ipilimumab). Examples, of PD-1/PD-L1 blockade inhibitors for use in the disclosed bioresponsive hydrogel matrixes can include any PD-1/PD-L1 blockade inhibitor known in the art, including, but not limited to nivolumab, pembrolizumab, pidilizumab, atezolizumab, avelumab, durvalumab, and BMS-936559). Thus, in one aspect, disclosed herein are methods of treating, preventing, inhibiting, and/or reducing a cancer and/or metastasis in a subject comprising administering to the subject a bioresponsive hydrogel matrix comprising a CD47/SIRPα inhibitor (such as, for example, Hu5F9-G4, CV1, B6H12, 2D3, CC-90002, and TTI-621) and an immune checkpoint blockade inhibitor; wherein the blockade inhibitor is a PD-1/PD-L1 blockade inhibitor such as, for example, nivolumab, pembrolizumab, pidilizumab, atezolizumab, avelumab, durvalumab, and BMS-936559; or a CTLA-4/B7-1/2 inhibitor such as, for example, Ipilimumab. It is understood and herein contemplated that the bioresponsive hydrogel matrix can be designed to incorporate 1, 2, 3, 4, 5, 6, 7, 8, 9, 10, 11, 12, 13, 14, or 15 blockade inhibitors simultaneously.

In one aspect, the disclosed methods of treating, preventing, inhibiting, and/or reducing a cancer and/or metastasis comprising administering to a subject any of the therapeutic agent delivery vehicles or pharmaceutical compositions and bioresponsive hydrogel matrixes disclosed herein, including but not limited to bioresponsive hydrogel matrixes comprising a CD47/SIRPα inhibitor (such as, for example, Hu5F9-G4, CV1, B6H12, 2D3, CC-90002, and TTI-621) and an immune checkpoint blockade inhibitor) can comprise administration of the pharmaceutical compositions or bioresponsive hydrogel matrixes at any frequency appropriate for the treatment of the particular cancer in the subject. For example, pharmaceutical compositions and/or bioresponsive hydrogel matrixes can be administered to the patient at least once every 12, 14, 16, 18, 20, 22, 24, 26, 28, 30, 32, 34, 36, 38, 40, 42, 44, 46, 48 hours, once every 3, 4, 5, 6, 7, 8, 9, 10, 11, 12, 13, 14, 15, 16, 17, 18, 19, 20, 21, 22, 23, 24, 25, 26, 27, 28, 29, 30, 31 days, once every 2, 3, 4, 5, 6, 7, 8, 9, 10, 11, or 12 months. In one aspect, the pharmaceutical compositions and/or bioresponsive hydrogel matrixes are administered at least 1, 2, 3, 4, 5, 6, 7 times per week.

As disclosed herein the bioresponsive hydrogel matrix scaffold can be designed to release any CD47/SIRPα inhibitor, an immune checkpoint blockade inhibitor, and/or additional anti-cancer agent encapsulated in the hydrogel as the degradation of the hydrogel occurs in response to factors in the tumor microenvironment. Accordingly disclosed herein are methods of treating, preventing, inhibiting, and/or reducing a cancer and/or metastasis in a subject wherein the bioresponsive hydrogel matrix comprises a bioresponsive scaffold that releases the CD47/SIRPα inhibitor, immune checkpoint blockade inhibitor, and/or any further encapsulated anti-cancer agent into a tumor microenvironment upon exposure to factors within the microenvironment. In one aspect, the bioresponsive hydrogel comprises a reactive oxygen species (ROS) degradable hydrogel. It is understood and herein contemplated that the release of the CD47/SIRPα inhibitor, immune checkpoint blockade inhibitor, and/or any further encapsulated anti-cancer agent by the bioresponsive hydrogel matrix into a tumor microenvironment is affected by the microenvironment. In one aspect, disclosed herein are methods of treating, preventing, inhibiting, and/or reducing a cancer and/or metastasis in a subject, wherein the bioresponsive hydrogel matrix releases the CD47/SIRPα inhibitor, immune checkpoint blockade inhibitor, and/or any further encapsulated anti-cancer agent into the tumor microenvironment for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days.

In one aspect, the amount of the pharmaceutical compositions and/or bioresponsive hydrogel matrixes disclosed herein which are administered to the subject for use in the disclosed methods can comprise any amount appropriate for the treatment of the subject for the particular cancer as determined by a physician. For example, the amount of the pharmaceutical compositions and/or bioresponsive hydrogel matrix can be from about 10 mg/kg to about 100 mg/kg. For example, the amount of the pharmaceutical compositions, bioresponsive hydrogels administered can be at least 10 mg/k, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, or 100 mg/kg. Accordingly, in one aspect, disclosed herein are methods of treating a cancer in a subject, wherein the dose of the administered pharmaceutical compositions and/or bioresponsive hydrogel matrix is from about 10 mg/kg to about 100 mg/kg.

As noted above, it is understood and herein contemplated that the disclosed methods of treating, preventing, inhibiting, and/or reducing a cancer and/or metastasis in a subject can further comprise the administration of any anti-cancer agent that would further aid in the reduction, inhibition, treatment, and/or elimination of the cancer and/or metastasis (such as, for example, gemcitabine). Anti-cancer agents that can be used in the disclosed bioresponsive hydrogels or as an additional therapeutic agent in addition to the disclosed pharmaceutical compositions, and/or bioresponsive hydrogel matrixes for the methods of reducing, inhibiting, treating, and/or eliminating a cancer and/or metastasis in a subject disclosed herein can comprise any anti-cancer agent known in the art, the including, but not limited to Abemaciclib, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin Chlorambucil), Amifostine, Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avastin (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Campath (Alemtuzumab), Camptosar, (Irinotecan Hydrochloride), Capecitabine, CAPDX, Carac (Fluorouracil—Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil—Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista, (Raloxifene Hydrochloride), Evomela (Melphalan Hydrochloride), Exemestane, 5-FU (Fluorouracil Injection), 5-FU (Fluorouracil—Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil—Topical), Fluorouracil Injection, Fluorouracil—Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and, Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq, (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil—Topical), Topotecan Hydrochloride, Toremifene, Torisel (Temsirolimus), Tositumomab and Iodine I 131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and/or Zytiga (Abiraterone Acetate).

The disclosed compositions can be used to treat any disease where uncontrolled cellular proliferation occurs such as cancers and metastasis, including, but not limited to cancers with low PD-L1 expression or a non-immunogenic cancers. A representative but non-limiting list of cancers that the disclosed compositions can be used to treat is the following: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, skin cancer, liver cancer, melanoma, squamous cell carcinomas of the mouth, throat, larynx, and lung, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon cancer, rectal cancer, prostatic cancer, or pancreatic cancer.

### D. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1: Reactive Oxygen Species-Responsive Protein Complex of aPD1 and aCD47 Antibodies for Enhanced Immunotherapy

a) Formation of Bioresponsive Antibody Complexes

The ROS-responsive antibody complex was obtained by crosslinking aPD1 and aCD47 via an ROS-responsive cross-linker: bis-N-hydroxy succinimide (NHS) modified 2,2′-[Propane-2,2-diylbis(thio)]diacetic acid (NHS-IE-NHS). Briefly, this protein complex was prepared via two steps: 1) aPD1 and albumin were mixed with NHS-IE-NHS to form aPD1 (albumin) core complex; 2) aCD47, additional albumin, and NHS-IE-NHS were added to allow coating of aCD47 (albumin) on the pre-synthesized aPD1 (albumin) core. The obtained aPD1@aCD47 complex showed a high antibody incorporation efficiency (˜90%). The average diameter of the core complex was 96 nm, and the final core-shell complex showed an increased size of 220 nm (FIG. 1B and C). The elemental mapping further validated the core-shell distribution of aPD1 (calcium-chelated) and aCD47 (gadolinium-chelated) in the protein complexes (FIG. 1D).

To verify the ROS-responsive dissociation behavior of the complex, the protein complexes were dissolved in phosphate-buffered saline (PBS) containing 500 μM H₂O₂. The dissociation of the complexes was observed via dynamic light scattering and TEM imaging (FIG. 1E). The release profiles of aPD1 and aCD47 were quantified using an enzyme-linked immunosorbent assay (ELISA). Consistent with expectations, aPD1 and aCD47 were released from the complex in the PBS solution containing H₂O₂, while a minimal amount of antibodies was released in pure PBS solution (FIG. 1F). As expected, aCD47 was released first, followed by the release of aPD1. This distinct release behavior of aPD1 and aCD47 facilitated their respective roles in the TME. Moreover, the ROS-sensitive linker can effectively scavenge H₂O₂ in the PBS solution (FIG. 1G).

b) ROS Scavenging Effect of Complex

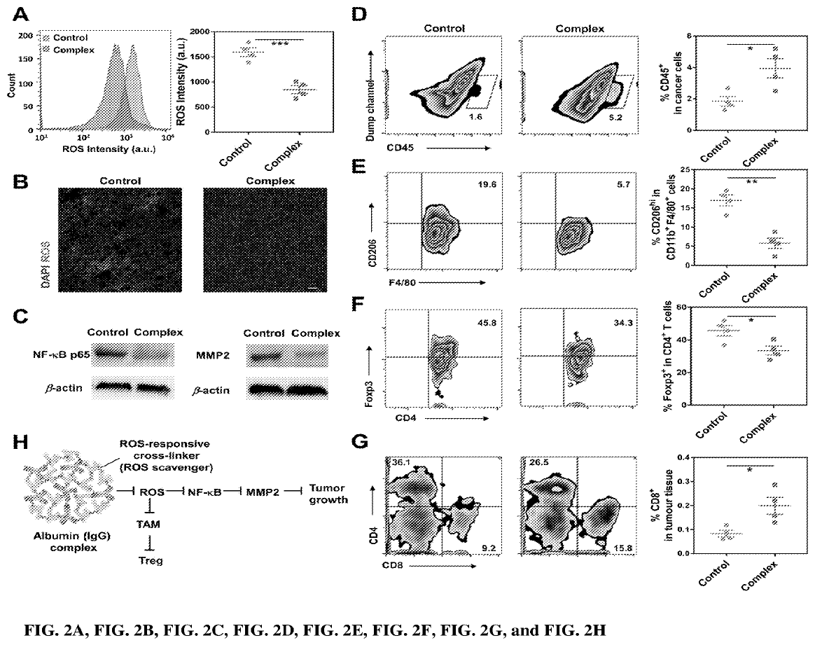

The ROS level is elevated in cancer during tumor development, which is usually associated with immunosuppressive TME, increasing the potential of tumor migration, invasion, and resistance. Considering the ability of the complex to capture ROS, the ROS level and immune responses of different immune cells was studied in the TME. As expected, the ROS level in the TME was significantly decreased after intertumoral (i.t.) injection of the blank complex (formed by IgG antibodies) (FIG. 2A and B). The ROS sensitive signal and redox-sensitive transcription factor, NF-κB, are known to motivate aberrant cancer cell proliferation and elevate matrix metalloproteinase (MMP) levels, promoting the invasive and metastatic process of tumors. Hence, the expression of NF-κB and MMP-2 was also examined in the tumor after treatment with the ROS sensitive blank complexes. Compared with the control group (untreated), obvious down-regulation of both NF-κB and MMP-2 was observed in the tumor (FIG. 2C).

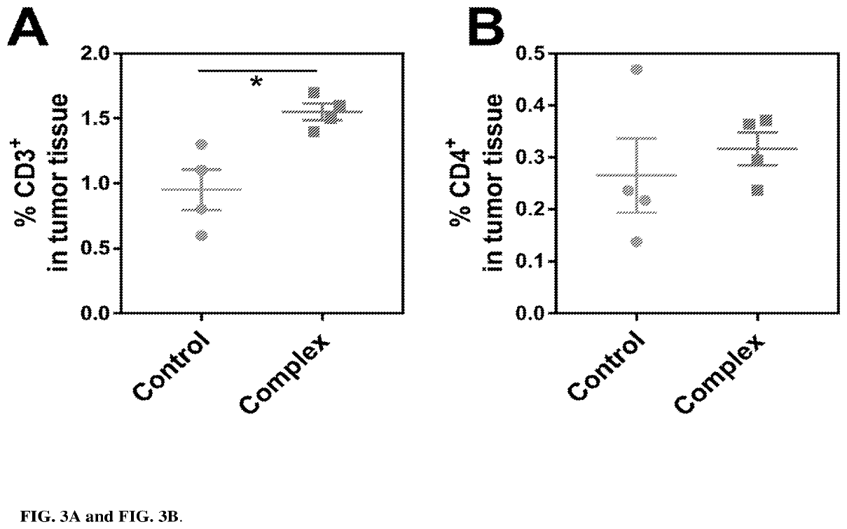

To further investigate the immune effects mediated by oxidative stress after depriving ROS using a blank complex, populations of different immune cells including tumor-associated macrophages (TAMs), myeloid-derived suppressor cells (MDSCs), and regulatory T cells (Tregs) were examined. While MDSCs (CD45⁺CD11b⁺Gr-1⁺) were not obviously decreased, a significant reduction of M2 type TAMs (CD45⁺CD11b⁺F4/80⁺CD206^{hi}) and Tregs (CD3⁺CD4⁺Foxp3⁺) was observed in the tumor injected with the blank complex (FIGS. 2D, 2E, and 2F). Furthermore, the percentage of tumor-infiltrating lymphocytes (CD3⁺, TILs) and cytotoxic T lymphocytes (CD3⁺CD8⁺, CTLs) was slightly increased in the tumor (FIG. 2G and FIG. 3). Taken together, scavenging of ROS in the TME using the ROS responsive complex can inhibit the expression of NF-κB and MMP-2, reduce immunosuppressive cells, and enhance the infiltration of effective T cells (FIG. 2H).

c) Immune Response Induced by CD47 Blockade

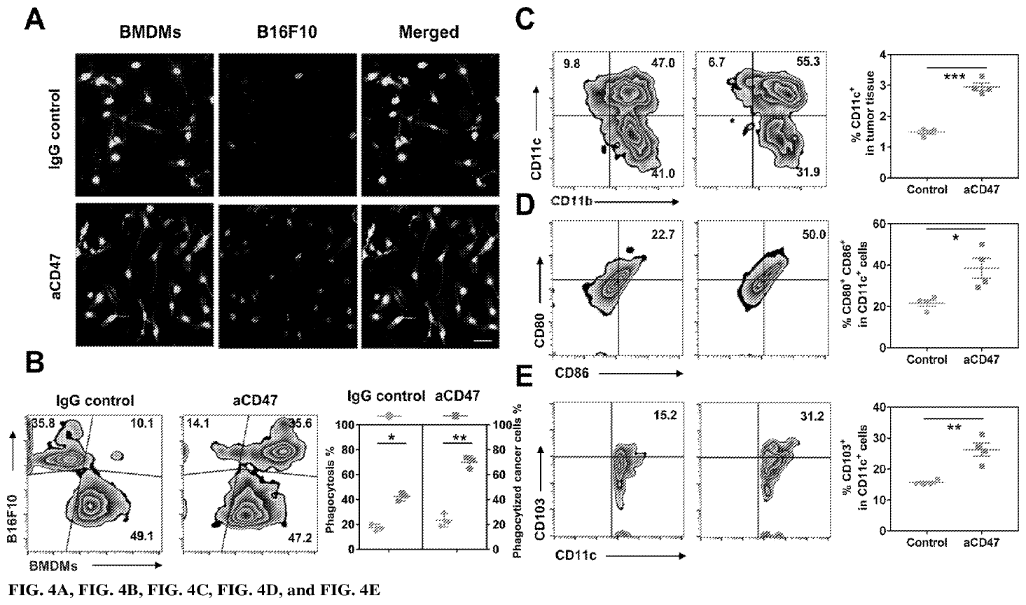

Innate immune cells including dendritic cells (DC) and macrophages play an important role in the initiation of the adaptive immune system via phagocytosis and presentation of antigens. However, cancer cells can usually escape from phagocytosis by upregulating the expression of CD47, a “don't eat me” signal. To verify that aCD47 can promote the phagocytosis of cancer cells by macrophages, the interaction between cancer cells and macrophages was first studied in vitro. Bone marrow-derived macrophages (BMDMs) labeled with green fluorescence signals were incubated with red fluorescence labeled B16F10 cells, which had been pre-incubated with IgG or aCD47 antibodies. Compared with IgG treated B16F10 cells, more cancer cells pre-incubated with aCD47 were phagocytosed by BMDMs (FIGS. 4A and 4B). To verify the ability of aCD47 to activate the antitumor immune responses, aCD47 complexes were i.t. injected into the tumor. More phagocytic cells including macrophages and DCs infiltrated into the tumor (FIG. 4C). To further assess immune responses after aCD47 treatment, the DC stimulation was studied by flow cytometry. Significantly increased DC maturation (CD80⁺CD86⁺) and increased percentage of CD103⁺ DCs were observed, which are critical for antigen transportation, T-cell activation and expansion, and intact antitumor immunity (FIGS. 4D and 4E).

d) Complex Extending the Release of Immunomodulatory Payloads

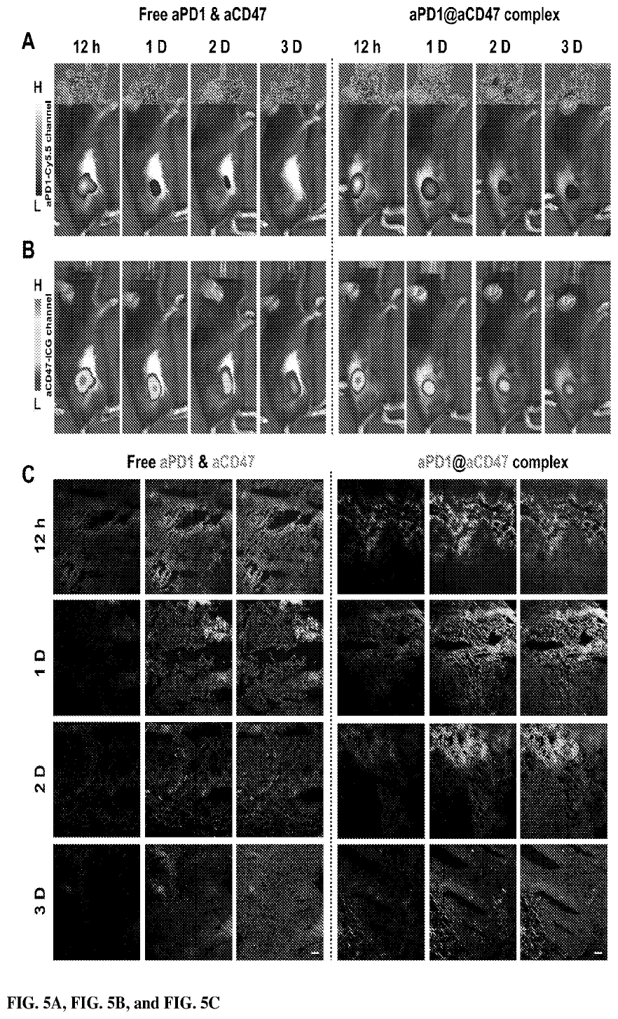

Considering the unique core-shell structure of the anti-body complexes, aCD47 on the surface of complexes can bind to the cancer cells, thus prolonging the retention of antibodies in the tumor. To verify this, the retention of immunomodulatory antibodies was studied in vivo. aPD1 and aCD47 were separately labeled with cyanine 5.5 (aPD1-Cy5.5) and indocyanine green (aCD47-ICG). Then, mice were injected with ‘Free aPD1 & aCD47’ or ‘aPD1@aCD47 complexes’ formulations in the tumor site and monitored using in vivo fluorescence imaging system at different time points after injection. It was observed that signals from free aPD1-Cy5.5 diminished quickly in the following three days, demonstrating the rapid diffusion of aPD1 out of the tumor, whereas aPD1-Cy5.5 encapsulated in the core of the complex still exhibited observed aPD1 signals in the tumor (FIG. 5A). The signals of aCD47 were comparable in two groups, substantiating the efficient binding between aCD47 and cancer cells, which also promoted the retention of complexes (FIG. 5B). Moreover, the prolonged retention of aPD1 in the tumor was further confirmed by the confocal imaging of the tumor sections, in which stronger red signals of aPD1 were observed in mice injected with aPD1@aCD47 complexes (FIG. 5C).

e) Antitumor Efficacy of aPD1@aCD47 Complexes In Vivo

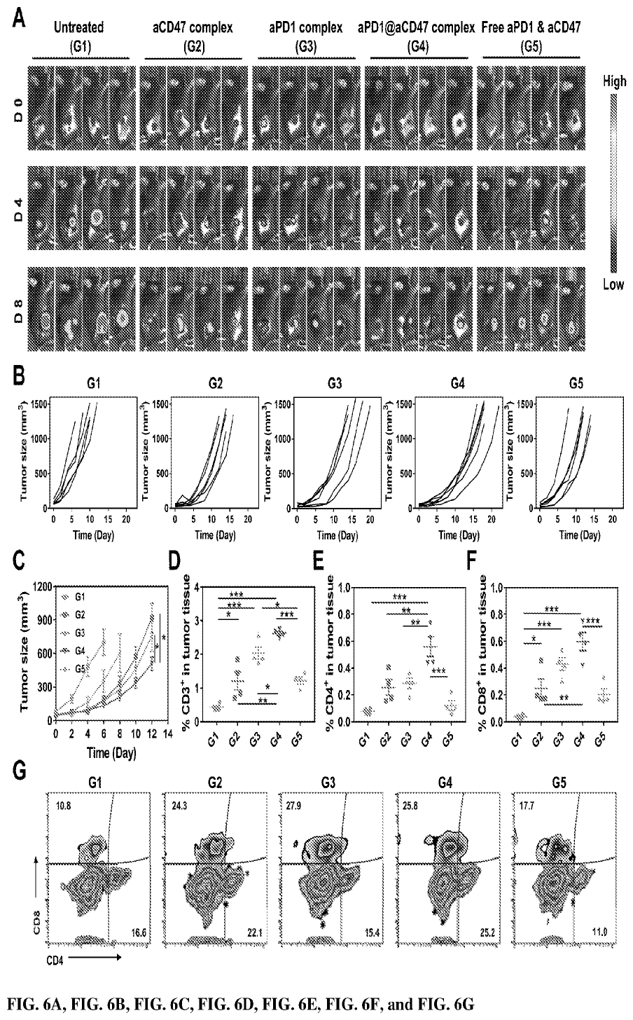

Then, the antitumor activity of aPD1@aCD47 complexes-based combination therapy was assessed in vivo. C57BL6 mice bearing melanoma B16F10 tumors were randomly divided into five groups: Untreated (G1), aPD1 complexes (aPD1 in both core and shell, 100 μg per mouse) (G2), aCD47 complexes (aCD47 in both core and shell, 100 μg per mouse) (G3), aPD1@aCD47 complexes (aCD47 in the shell, 50 μg per mouse; aPD1 in the core, 50 μg per mouse) (G4), and free aPD1 & aCD47 (aCD47: 50 μg per mouse, aPD1: 50 μg per mouse). In the following days, the growth of the tumor was monitored by the bioluminescence imaging and measured by a traditional caliper (FIGS. 6A, 6B, and 6C). The tumor growth in mice treated with aPD1@aCD47 complexes was significantly slower than the other four groups. Compared with aPD1 complexes or aCD47 complexes treated groups, an obvious synergistic effect was achieved by aPD1@aCD47 complexes treatment. Notably, the growth of tumor in the free antibodies treated group was only inhibited in the first two days, owing to the rapid diffusion of free aPD1. Moreover, the body weights of mice in different groups were not affected.

To investigate the immune responses in tumors after different treatments, tumors were collected and analyzed by the flow cytometry and immunofluorescence imaging five days after treatment. Compared with the untreated group, more TILs (CD3⁺ cells) infiltrated into the tumor of mice than those in the other four treated groups. Moreover, the absolute number of CD4⁺ T cells and CD8⁺ T cells in the tumor was significantly increased after treated with aPD1@aCD47 complexes (FIGS. 6D, 6E, 6F, and 6G). The immunofluorescence imaging visually indicated that there were more CD8⁺ T cells inflated into the tumor treated with aPD1@aCD47 complexes. Collectively, these observations indicated that the combination therapy using aPD1@aCD47 complexes triggered enhanced T cell-mediated anti-cancer immune response.

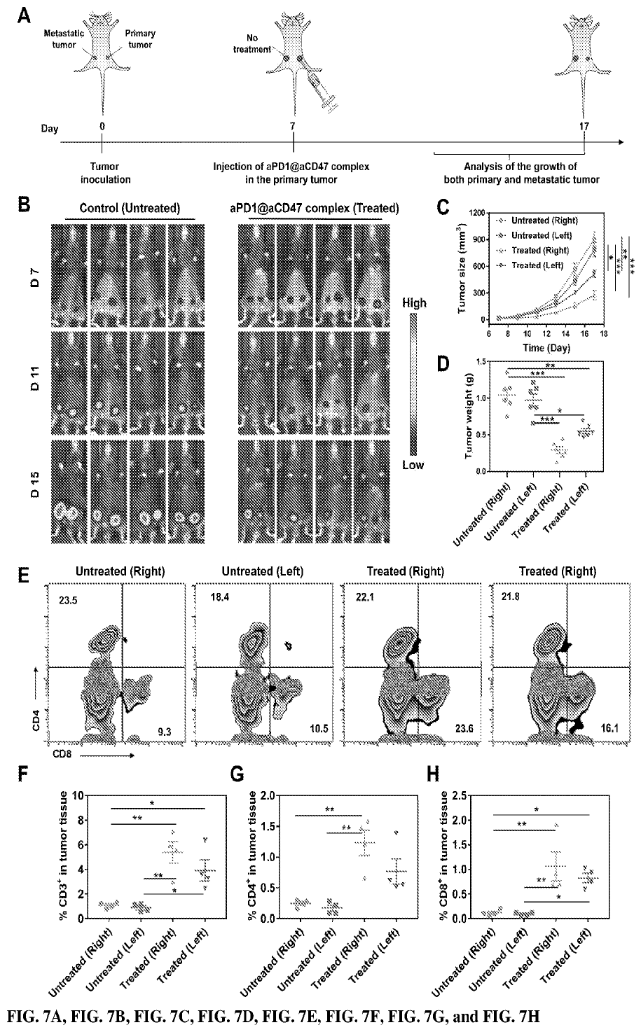

Next, it was investigated whether the local injection of aPD1@aCD47 complexes can induce systemic immune responses to inhibit cancer metastasis. B16F10 tumor cells were inoculated on both right and left flanks of each mouse. The tumor in the right flank as the primary tumor was injected with aPD1@aCD47 complexes, and the distant tumor on the opposite site received no treatment to mimic cancer metastasis (FIG. 7A). The bioluminescence signal from the tumor and the size of the tumor significantly decreased in the mice injected with aPD1@aCD47 complexes. Notably, for mice injected with aPD1@aCD47 complexes in their primary tumors, their distant tumors were also effectively inhibited (FIGS. 7B and 7C). Consistent with these results, the weight of primary and distant tumors in aPD1@aCD47 complexes treated mice was also significantly lower, which were paralleled with an increase in the percentage of CD3⁺ TIL (FIG. 7D, 7E, 7F). Additionally, the intertumoral percentage of CD8⁺ and CD4⁺ T cells also significantly increased both in primary and distant tumors in the treated mice (FIGS. 7G and 7H). Taken together, these results indicated that the local injection of aPD1@aCD47 complexes can active systemic immune responses to inhibit potential metastasis.

f) Discussion

In this study, a protein-based complex containing aPD1 in the core and aCD47 in the shell using ROS-sensitive linkers for enhanced immune checkpoint blockade was engineered. ROS produced in the TME usually play a vital role as a signaling messenger in the immune system, which is associated with the tumor-associated immunosuppression and the dysfunction of T cells. It was shown herein that synergistic therapeutic efficacy can be achieved by the bioresponsive protein complex. Considering the abundant ROS in the TME and the unique core-shell structure, the aPD1@aCD47 complex can sequentially release aCD47 from the outer shell aPD1 from the inner core. The released aCD47 blocked the “don't eat me” signals in tumor cells, promoting the recognition of cancer cells by the innate immune systems and activating the T-cell immune responses. The further subsequently released aPD1 can blockade PD-1 on TIL, increasing alloreactive T cell population. The distribution of aCD47 on the surface of the protein complex, can prolong the retention of antibodies in the tumor. Moreover, the ROS-responsive linkers not only contribute to the controlled release of antibodies, but also act as scavenger of ROS to reverse the immunosuppressive TME. The down-regulation of NF-κB and MMP-2 expression, reduced immunosuppressive cells including TAMs and Tregs, and enhanced the infiltration of effective T cells in the tumor was observed. Furthermore, the local treatment of the ROS-responsive protein complex can generate systemic antitumor immune responses that not only inhibit the primary tumor growth, but also prevent the potential of cancer metastasis.

In summary, the bioresponsive protein complex can effectively reverse the immunosuppressive TME and promote immune checkpoint blockade. The unique core-shell distribution of aCD47 and aPD1 in the complexes prolonged the retention of antibodies and realized the sequentially release of antibodies in the tumor site. Nevertheless, parameters associated with the protein complex need further optimization, such as the optimization of the ROS-responsive liner, as well as the percentage of aCD47 and aPD1.

g) Materials and Methods

(1) Preparation and Characterization of aPD1@aCD47 Complexes.

To synthesize the ROS-responsive cross-linker, 2,2′-[Propane-2,2-diylbis(thio)]diacetic acid (5.0 mg, 1 equiv), EDC (6.9 mg, 2 equiv) and NHS (5.1 mg, 2 equiv) were mixed in dimethyl sulfoxide (DMSO) and stirred for 6 hours at room temperature. To obtain the aPD1 core complexes, albumin from mouse serum (20 equiv.) and aPD1 (1 equiv.) were mixed in phosphatebuffered saline (PBS), and then the ROS-responsive cross-linker (200 equiv.) in DMSO was added slowly. The mixture was stirred overnight at 4° C. The obtained aPD1 core complexes were purified after centrifugation at 20000 rpm to remove free albumin or antibodies. Afterwards, additional albumin (20 equiv.), aCD47 (1 equiv.), and ROS-responsive cross-linkers (200 equiv.) were added into aPD1 complexes solution and stirred overnight at 4° C. The obtained aPD1@aCD47 complexes were purified after centrifugation at 20000 rpm to remove free albumin or antibodies. The control blank complexes were prepared using IgG from rat to replace relative antibodies following the same procedure.

(2) Characterization.

The size distribution and morphology of aPD1 complexes and aPD1@aCD47 complexes were measured by dynamic laser scattering (DLS) and TEM (JEOL 2000FX), respectively. The distribution of antibodies in the complexes was characterized using an analytical TEM (Titan) (aCD47 and aPD1 were chelated gadolinium and calcium, respectively. The amount of different antibodies (IgG from rat serum indicates aCD47 and IgG from rabbit serum indicates aPD1) conjugated in the complexes was measured by ELISA (rat IgG total ELISA kit, eBioscience, cat. no. 88-50490-22; rabbit IgG total ELISA kit, Thermo Fisher, cat. no. 15137).

(3) In Vitro Phagocytosis Assay.

Bone marrow derived macrophages (BMDMs) separated from C57BL/6 mice were stained with CellTracker Green (C7025, Thermo-Fisher Scientific), and B16F10 cells were stained with CellTracker DeepRed (C34565, Thermo-Fisher Scientific, USA). Cancer cells were blocked with IgG or aCD47 and then co-cultured with macrophages in the serum free medium. After incubation for 2 h at 37° C., confocal microscopy (Zeiss LSM 710) and CytoFLEX flow cytometry (Beckman) were used to study the phagocytosis behavior of cancer cells by macrophages.

(4) In Vivo Tumor Models and Treatment.

To measure the combination therapeutic effects of aPD1@aCD47 complexes, fLuc-B16F10 melanoma cells (lx 10⁶) were subcutaneously (s.c.) injected into the right flank of each C57BL/6 mouse. Seven days later, mice were divided into five groups (n=6) randomly. The mice were intratumorally (i.t.) injected with different formations including aCD47 complexes (aCD47:100 μg per mouse), aPD1 complexes (aPD1:100 μg per mouse), aPD1@aCD47 complexes (aCD47:50 μg per mouse, aPD1:50 μg per mouse), free aPD1 & free aCD47 (aCD47:50 μg per mouse, aPD1:50 μg per mouse). For a metastatic tumor model, a total of 1×10⁶fLuc-B16F10 melanoma cells were s.c. injected into both flanks of each C57BL/6 mouse. A week later, aPD1@aCD47 complexes (aCD47:50 μg per mouse, aPD1:50 μg per mouse) were i.t. injected into the tumor on the right flank of each mouse. The volume of tumor was measured and calculated according to the following formula: width²×length×0.5. The growth of tumor also was observed by an in vivo imaging instrument (IVIS) Spectrum System (Perkin Elmer Ltd). Ten mins after intraperitoneal injection of d-luciferin (Thermo Scientific™ Pierce™) into each mouse (0.15 mg/g), the mouse was imaged with 5 min exposure time. Animals were euthanized when showing signs of imperfect health or when the size of their tumors exceeded 1.5 cm³.

(5) Materials, Cell Lines, and Animals.

All chemicals including 2,2′-[Propane-2,2-diylbis(thio)]diacetic acid, 1-Ethyl-3-(3-dimethylaminopropyl)-carbodiimide (EDC), N-Hydroxysuccinimide (NHS) were purchased from Sigma-Aldrich. Albumin from mouse serum, Immunoglobulin G (IgG) from rat serum and IgG from rabbit serum were purchased from Sigma-Aldrich. Anti-CD47 antibody (aCD47) (Cat. #127518, Clone: miap301) and anti-PD1 antibody (aPD1) (Cat. #135233, Clone: 29F.1A12) were purchased from Biolegend Inc. The murine B16F10 melanoma cell line was purchased from the UNC tissue culture facility. B16F10-luc cells were gifts from Dr. Leaf Huang at University of North Carolina at Chapel Hill. Cells were cultured in the Dulbecco's Modified Eagle Medium (Gibco, Invitrogen) containing 100 U/mL penicillin (Invitrogen) and 10% fetal bovine serum (Invitrogen, Carlsbad, Calif.) at 37° C. in 5% CO₂. Female C57BL/6 mice (6-10 weeks) were purchased from Jackson Lab. All mouse studies were carried out following the protocols approved by the Institutional Animal Care and Use Committee at the University of North Carolina at Chapel Hill and North Carolina State University.

(6) Flow Cytometry.

To investigate different immune cells in the tumor, tumors collected from mice after different treatments were cut into small pieces and homogenized to form single cells in cold staining buffer. Cells were stained with fluorescence-labeled antibodies CD45 (Biolegend, cat. no. 103108, Clone: 30-F11), CD11b (Biolegend, cat. no. 101212, Clone: M1/70), F4/80 (Biolegend, cat. no. 123128, Clone: BM8), CD80 (Biolegend, cat. no. 104708, Clone: 16-10A1), CD206 (Biolegend, cat. No. 141706, Clone: C068C2), Gr-1 (Biolegend, cat. no. 108408, Clone: RB6-8C5), CD3 (Biolegend, cat. no. 100236, Clone: 17A2), CD8 (Biolegend, cat. no. 100734, Clone: 53-6.7), CD4 (Biolegend, cat. no. 100406, Clone: GK1.5), Foxp3 (Biolegend, cat. no. 126404, Clone: MF-14), CD11c (Biolegend, cat. no. 117310, Clone: N418), CD86 (Biolegend, cat. no. 105028, Clone: GL-1), and CD103 (Biolegend, cat. no. 121406, Clone: 2E7) following the manufacturers' instructions. All antibodies used here were diluted 200 times. The stained cells were measured on a CytoFLEX flow cytometer (Beckman) and analyzed by the FlowJo software package (version 10.0.7; TreeStar, USA, 2014).

(7) Immunofluorescence Staining.

Tumors were harvest from the mice in different groups and frozen in optimal cutting temperature (OCT) medium. Tumors were cut via a cryotome, mounted on slides and stained with CD8 (Abcam, cat. no. ab22378) primary antibody overnight at 4° C. In the following, fluorescently labelled goat anti-rat IgG (H+L; Thermo Fisher Scientific, cat. no. A18866) secondary antibody was added. The slides were recorded using a confocal microscope (Zeiss LSM 710). All these antibodies used in the experiments were diluted 200 times.

(8) Western Blotting.

Each sample with an equal amount of protein determined by Bicinchoninic Acid Protein Assay Kit (BCA) was mixed with an equal volume of 2× Laemmli buffer and boiled at 95° C. for 5 min. After accomplishment of gel electrophoresis and protein transformation, anti-NF-κB p65 antibody (Abcam, cat. no. ab237591), anti-MMP2 antibody (Abcam, cat. no. ab92536) and anti-β-actin antibody (Abcam, cat. no. ab8226) were used as primary antibodies according to the manufacturers' instructions. The secondary antibodies including goat anti-mouse antibody (Novus Biologicals, cat. no. NBP1-75151) and goat anti-rabbit antibody (Novus Biologicals, cat. no. NBP2-30348H) were used for these blots.

(9) Statistical Analysis.

All results are appeared as the mean±standard error of the mean (s.e.m.) as indicated. Tukey post-hoc tests and One-way analysis of variance (ANOVA) were used for multiple comparisons and two-tailed Student's t-test was used for two group comparison. Survival benefit was determined using a log-rank test. All statistical analyses were carried out by the Prism software package (PRISM 5.0; GraphPad Software, USA, 2007). The threshold for statistical significance was P<0.05.

### E. REFERENCES

A. D. Michaels, T. E. Newhook, S. J. Adair, S. Morioka, B. J. Goudreau, S. Nagdas, M. G. Mullen, J. B. Persily, T. N. Bullock, C. L. Slingluff, CD47 Blockade as an Adjuvant Immunotherapy for Resectable Pancreatic Cancer. Clin. Cancer Res. 24, 1415-1425 (2018).
A. Ribas, J. D. Wolchok, Cancer immunotherapy using checkpoint blockade. Science 359, 1350-1355 (2018).
B. Edris, K. Weiskopf, A. K. Volkmer, J.-P. Volkmer, S. B. Willingham, H. Contreras-Trujillo, J. Liu, R. Majeti, R. B. West, J. A. Fletcher, Antibody therapy targeting the CD47 protein is effective in a model of aggressive metastatic leiomyosarcoma. Proc. Nat. Acad. Sci. U.S.A 109, 6656-6661 (2012).
C. Nathan, A. Cunningham-Bussel, Beyond oxidative stress: an immunologist's guide to reactive oxygen species. Nat. Rev. Immunol. 13, 349 (2013).
C. Wang, J. Wang, X. Zhang, S. Yu, D. Wen, Q. Hu, Y. Ye, H. Bomba, X. Hu, Z. Liu, In situ formed reactive oxygen species-responsive scaffold with gemcitabine and checkpoint inhibitor for combination therapy. Sci. Transl. Med. 10, eaan3682 (2018).
C. Wang, W. Sun, Y. Ye, Q. Hu, H. N. Bomba, Z. Gu, In situ activation of platelets with checkpoint inhibitors for post-surgical cancer immunotherapy. Nat. Biomed. Eng. 1, 0011 (2017).
D. M. Pardoll, The blockade of immune checkpoints in cancer immunotherapy. Nat. Rev. Cancer 12, 252 (2012).
D. Mittal, M. M. Gubin, R. D. Schreiber, M. J. Smyth, New insights into cancer immunoediting and its three component phases—elimination, equilibrium and escape. Curr. Opin. Immunol. 27, 16-25 (2014).
E. D. Kwon, B. A. Foster, A. A. Hurwitz, C. Madias, J. P. Allison, N. M. Greenberg, M. B. Burg, Elimination of residual metastatic prostate cancer after surgery and adjunctive cytotoxic T lymphocyte-associated antigen 4 (CTLA-4) blockade immunotherapy. Proc. Nat. Acad. Sci. U.S.A. 96, 15074-15079 (1999).
E. I. Buchbinder, F. S. Hodi, Melanoma in 2015: Immune-checkpoint blockade—Durable cancer control. Nat. Rev. Clin. Oncol. 13, 77 (2016).
E. V. Yang, A. K. Sood, M. Chen, Y. Li, T. D. Eubank, C. B. Marsh, S. Jewell, N. A. Flavahan, C. Morrison, P.-E. Yeh, Norepinephrine up-regulates the expression of vascular endothelial growth factor, matrix metalloproteinase (MMP)-2, and MMP-9 in nasopharyngeal carcinoma tumor cells. Cancer Res. 66, 10357-10364 (2006).
G.-Y. Liou, P. Storz, Reactive oxygen species in cancer. Free Radic. Res. 44, 479-496 (2010).
H. Tang, Y. Wang, L. K. Chlewicki, Y. Zhang, J. Guo, W. Liang, J. Wang, X. Wang, Y.-X. Fu, Facilitating T cell infiltration in tumor microenvironment overcomes resistance to PD-L1 blockade. Cancer cell 29, 285-296 (2016).
H. Tao, P. Qian, F. Wang, H. Yu, Y. Guo, Targeting CD47 Enhances the Efficacy of Anti-PD-1 and CTLA-4 in an Esophageal Squamous Cell Cancer Preclinical Model. Oncology Research Featuring Preclinical and Clinical Cancer Therapeutics 25, 1579-1587 (2017).
H. Wang, D. J. Mooney, Biomaterial-assisted targeted modulation of immune cells in cancer treatment. Nat. Mater 17, 761-772 (2018).
J. Naidoo, D. Page, B. Li, L. Connell, K. Schindler, M. Lacouture, M. Postow, J. Wolchok, Toxicities of the anti-PD-1 and anti-PD-L1 immune checkpoint antibodies. Ann. Oncol. 26, 2375-2391 (2015).
J. S. Stamler, Redox signaling: nitrosylation and related target interactions of nitric oxide. Cell 78, 931-936 (1994).
J. T. Sockolosky, M. Dougan, J. R. Ingram, C. C. M. Ho, M. J. Kauke, S. C. Almo, H. L. Ploegh, K. C. Garcia, Durable antitumor responses to CD47 blockade require adaptive immune stimulation. Proc. Nat. Acad. Sci. U.S.A 113, 2646-2654 (2016).
K. D. Moynihan, C. F. Opel, G. L. Szeto, A. Tzeng, E. F. Zhu, J. M. Engreitz, R. T. Williams, K. Rakhra, M. H. Zhang, A. M. Rothschilds, Eradication of large established tumors in mice by combination immunotherapy that engages innate and adaptive immune responses. Nat. Med. 22, 1402 (2016).
K. M. Mahoney, P. D. Rennert, G. J. Freeman, Combination cancer immunotherapy and new immunomodulatory targets. Nat. Rev. Drug Discov. 14, 561 (2015).
L. Tang, Y. Zheng, M. B. Melo, L. Mabardi, A. P. Castaño, Y.-Q. Xie, N. Li, S. B. Kudchodkar, H. C. Wong, E. K. Jeng, Enhancing T cell therapy through TCR-signaling-responsive nanoparticle drug delivery. Nat. Biotechnol. 36, 707 (2018).
M. A. Meyer, J. M. Baer, B. L. Knolhoff, T. M. Nywening, R. Z. Panni, X. Su, K. N. Weilbaecher, W. G. Hawkins, C. Ma, R. C. Fields, Breast and pancreatic cancer interrupt IRF8-dependent dendritic cell development to overcome immune surveillance. Nat. Commun. 9, 1250 (2018).
M. Diehn, R. W. Cho, N. A. Lobo, T. Kalisky, M. J. Dorie, A. N. Kulp, D. Qian, J. S. Lam, L. E. Ailles, M. Wong, Association of reactive oxygen species levels and radioresistance in cancer stem cells. Nature 458, 780 (2009).
M. Egeblad, Z. Werb, New functions for the matrix metalloproteinases in cancer progression. Nat. Rev. Cancer 2, 161 (2002).
M. F. Sanmamed, L. Chen, A Paradigm Shift in Cancer Immunotherapy: From Enhancement to Normalization. Cell 175, 313-326 (2018).
M. N. Wykes, S. R. Lewin, Immune checkpoint blockade in infectious diseases. Nat. Rev. Immunol. 18, 91 (2018).
M. P. Chao, A. A. Alizadeh, C. Tang, J. H. Myklebust, B. Varghese, S. Gill, M. Jan, A. C. Cha, C. K. Chan, B. T. Tan, Anti-CD47 antibody synergizes with rituximab to promote phagocytosis and eradicate non-Hodgkin lymphoma. Cell 142, 699-713 (2010).
N. G. Ring, D. Herndler-Brandstetter, K. Weiskopf, L. Shan, J.-P. Volkmer, B. M. George, M. Lietzenmayer, K. M. McKenna, T. J. Naik, A. McCarty, Anti-SIRPα antibody immunotherapy enhances neutrophil and macrophage antitumor activity. Proc. Nat. Acad. Sci. U.S.A 114, 10578-10585 (2017).
N. S. Katheder, R. Khezri, F. O'Farrell, S. W. Schultz, A. Jain, M. M. Rahman, K. O. Schink, T. A. Theodossiou, T. Johansen, G. Juhász, Microenvironmental autophagy promotes tumour growth. Nature 541, 417 (2017).
P. C. Brooks, S. Stromblad, L. C. Sanders, T. L. von Schalscha, R. T. Aimes, W. G. Stetler-Stevenson, J. P. Quigley, D. A. Cheresh, Localization of matrix metalloproteinase MMP-2 to the surface of invasive cells by interaction with integrin avβ3. Cell 85, 683-693 (1996).
P. Sharma, J. P. Allison, Immune checkpoint targeting in cancer therapy: toward combination strategies with curative potential. Cell 161, 205-214 (2015).
P. Sharma, J. P. Allison, The future of immune checkpoint therapy. Science 348, 56-61 (2015).
Q. Chen, C. Liang, X. Wang, J. He, Y. Li, Z. Liu, An albumin-based theranostic nano-agent for dual-modal imaging guided photothermal therapy to inhibit lymphatic metastasis of cancer post surgery. Biomaterials 35, 9355-9362 (2014).
Q. Chen, C. Wang, G. Chen, Q. Hu, Z. Gu, Delivery Strategies for Immune Checkpoint Blockade. Adv. Healthc. Mater., 1800424 (2018).
Q. Chen, C. Wang, X. Zhang, G. Chen, Q. Hu, H. Li, J. Wang, D. Wen, Y. Zhang, Y. Lu, In situ sprayed bioresponsive immunotherapeutic gel for post-surgical cancer treatment. Nat. Nanotechnol., (2018).
Q. Chen, L. Xu, C. Liang, C. Wang, R. Peng, Z. Liu, Photothermal therapy with immune-adjuvant nanoparticles together with checkpoint blockade for effective cancer immunotherapy. Nat. Commun. 7, 13193 (2016).
R. H. Vonderheide, CD47 blockade as another immune checkpoint therapy for cancer. Nat. Med. 21, 1122 (2015).
R. S. Riley, C. H. June, R. Langer, M. J. Mitchell, Delivery technologies for cancer immunotherapy. Nat. Rev. Drug Discov., 1 (2019).
S. B. Willingham, J.-P. Volkmer, A. J. Gentles, D. Sahoo, P. Dalerba, S. S. Mitra, J. Wang, H. Contreras-Trujillo, R. Martin, J. D. Cohen, The CD47-signal regulatory protein alpha (SIRPa) interaction is a therapeutic target for human solid tumors. Proc. Nat. Acad. Sci. U.S.A 109, 6662-6667 (2012).
S. I. Grivennikov, F. R. Greten, M. Karin, Immunity, inflammation, and cancer. Cell 140, 883-899 (2010).
S. L. Topalian, J. M. Taube, R. A. Anders, D. M. Pardoll, Mechanism-driven biomarkers to guide immune checkpoint blockade in cancer therapy. Nat. Rev. Cancer 16, 275 (2016).
S. R. Gordon, R. L. Maute, B. W. Dulken, G. Hutter, B. M. George, M. N. McCracken, R. Gupta, J. M. Tsai, R. Sinha, D. Corey, PD-1 expression by tumour-associated macrophages inhibits phagocytosis and tumour immunity. Nature 545, 495 (2017).
S. Subramanian, R. Parthasarathy, S. Sen, E. T. Boder, D. E. Discher, Species-and cell type-specific interactions between CD47 and human SIRPα. Blood 107, 2548-2556 (2006).
S. Yu, C. Wang, J. Yu, J. Wang, Y. Lu, Y. Zhang, X. Zhang, Q. Hu, W. Sun, C. He, Injectable Bioresponsive Gel Depot for Enhanced Immune Checkpoint Blockade. Adv. Mater., 1801527 (2018).
T. N. Schumacher, R. D. Schreiber, Neoantigens in cancer immunotherapy. Science 348, 69-74 (2015).
Y. Huang, Y. Ma, P. Gao, Z. Yao, Targeting CD47: the achievements and concerns of current studies on cancer immunotherapy. J. Thorac. Dis. 9, E168-E174 (2017).
Y. Lu, A. A. Aimetti, R. Langer, Z. Gu, Bioresponsive materials. Nat. Rev. Mater. 2, 16075 (2017).

1. A bioresponsive hydrogel matrix comprising a CD47/SIRPα inhibitor and an immune checkpoint blockade inhibitor.

2. The bioresponsive hydrogel matrix of claim 1, wherein the CD47/SIRPα inhibitor is selected from the group consisting of Hu5F9-G4, CV1, B6H12, 2D3, CC-90002, and TTI-621.

3. The bioresponsive hydrogel matrix of claim 1, wherein the immune checkpoint blockade inhibitor is a PD-1/PD-L1 blockade inhibitor.

4. The bioresponsive hydrogel matrix of claim 3, wherein the PD-1/PD-L1 blockade inhibitor is selected from the group consisting of nivolumab, pembrolizumab, pidilizumab, atezolizumab, avelumab, durvalumab, and BMS-936559.

5. The bioresponsive hydrogel matrix of claim 1, wherein the immune checkpoint blockade inhibitor is a CTLA-4/B7-1/2 blockade inhibitor.

6. The bioresponsive hydrogel matrix of claim 5, wherein the CTLA-4/B7-1/2 blockade inhibitor comprises Ipilimumab.

7. The bioresponsive hydrogel matrix of claim 1, wherein the hydrogel matrix comprises a reactive oxygen species (ROS) degradable hydrogel.

8. The bioresponsive hydrogel matrix of claim 1, wherein the bioresponsive hydrogel matrix comprises crosslinked albumin.

9. The bioresponsive hydrogel matrix of claim 1, wherein bioresponsive hydrogel matrix comprises an inner core and an outer shell; and wherein the CD47/SIRPα inhibitor is cross-linked to the outer shell and the immune checkpoint inhibitor is cross-linked to the inner core.

10. The bioresponsive hydrogel matrix of claim 1, wherein bioresponsive hydrogel matrix comprises an inner core and an outer shell; and wherein the CD47/SIRPα inhibitor is cross-linked to the inner core and the immune checkpoint inhibitor in cross-linked to the outer shell.

11. The bioresponsive hydrogel matrix of claim 9, wherein the CD47/SIRPα inhibitor and the immune checkpoint inhibitor are cross-linked to the bioresponsive hydrogel matrix by ROS-responsive cross-linker.

12. The bioresponsive hydrogel matrix of claim 11, wherein the cross-linker comprises bis-N-hydroxy succinimide (NHS) modified 2,2′-[Propane-2,2-diylbis(thio)]diacetic acid (NHS-LE-NHS).

13. A method of treating a cancer in a subject comprising administering to the subject the bioresponsive hydrogel matrix of claim 1.

14. A method of treating a cancer in a subject comprising administering to the subject a bioresponsive hydrogel matrix comprising a CD47/SIRPα inhibitor and an immune checkpoint blockade inhibitor.

15. The method of claim 14, wherein the CD47/SIRPα inhibitor is selected from the group consisting of Hu5F9-G4, CV1, B6H12, 2D3, CC-90002, and TTI-621.

16. The method of claim 14, wherein the blockade inhibitor is a PD-1/PD-L1 blockade inhibitor or a CTLA-4/B7-1/2 blockade inhibitor.

17. The method of claim 16, wherein the PD-1/PD-L1 blockade inhibitor is selected from the group consisting of nivolumab, pembrolizumab, pidilizumab, atezolizumab, avelumab, durvalumab, and BMS-936559.

18. (canceled)

19. The method of claim 16, wherein the CTLA-4/B7-1/2 blockade inhibitor comprises Ipilimumab.

20. The method of treating a cancer of claim 13, wherein the cancer is selected from the group consisting of lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, renal cancer, small cell lung cancer and non-small cell lung cancer, neuroblastoma, glioblastoma, ovarian cancer, pancreatic cancer, prostate cancer, skin cancer, liver cancer, melanoma, squamous cell carcinomas of the mouth, throat, larynx, and lung, colon cancer, cervical cancer, cervical carcinoma, breast cancer, epithelial cancer, genitourinary cancer, pulmonary cancer, esophageal carcinoma, head and neck carcinoma, large bowel cancer, hematopoietic cancers; testicular cancer; colon cancers, and rectal cancers.

21. The bioresponsive hydrogel matrix of claim 10, wherein the CD47/SIRPa inhibitor and the immune checkpoint inhibitor are cross-linked to the bioresponsive hydrogel matrix by ROS-responsive cross-linker.
